(19) United States
(12) Patent Application Publication
(10) Pub. No.: US 2023/0237607 A1
(43) Pub. Date: Jul. 27, 2023

(54) SYSTEM AND METHOD FOR DETERMINING AN INTERIM LIFE SAFETY MEASURE (ILSM) FOR ONE OR MORE SMOKE COMPARTMENTS IN A FACILITY

(71) Applicant: Healthcare Facility Compliance Corp, Woodcliff Lake, NJ (US)

(72) Inventors: Ilan Nachmany, Upper Saddle River, NJ (US); Sumer Mathur, Great Falls, VA (US)

(21) Appl. No.: 18/180,890

(22) Filed: Mar. 9, 2023

Related U.S. Application Data
(63) Continuation-in-part of application No. 16/190,908, filed on Nov. 14, 2018, now abandoned.

(51) Int. Cl. G06Q 50/26; G06Q 10/0635; G06Q 10/20

(52) U.S. Cl. CPC G06Q 50/265; G06Q 10/0635; G06Q 10/20

(57) ABSTRACT

A system and method for determining an interim life safety measure (ILSM) for one or more smoke compartments in the facility is disclosed. The system analyzes potential risk factors associated with individuals in one or more smoke compartments of facility, based on type of smoke compartments. Further, the system classifies assets associated with smoke compartments into risk levels, based on asset classes and/or location environment associated assets comprising risk levels. Additionally, system assigns risk assessment scores to inspection points corresponding to classified assets. Furthermore, the system determines ILSMs. The ILSM is health and safety measure to protect individuals at facility. Additionally, system creates maintenance work orders and/or corrective work orders, based on determined ILSMs. Further, system outputs, to user device, notifications corresponding to status of created maintenance work orders and/or corrective work orders. The notifications inform user to provide instructions for creating order and inform user to monitor status.

### CROSS REFERENCE TO RELATED APPLICATIONS

This application is a continuation-in-part of U.S. patent application Ser. No. 16/190,908, filed on Nov. 14, 2018, entitled “Enterprise Mobile, Cloud Based Health Care Risk Assessment System,” which claims the benefit of continuation-in-part of U.S. patent application having Ser. No. 16/184,891, filed on Nov. 8, 2018, and entitled “Enterprise Mobile, Cloud Based Health Care Compliance System,” which again claims the priority of U.S. provisional patent application having Ser. No. 62/583,453, filed on Nov. 8, 2017, all of which are hereby incorporated by reference in their entirety.

### TECHNICAL FIELD

Embodiments of present disclosure generally relate to a risk assessment of a facility for a health care compliance and more particularly embodiments of present disclosure relate to a system and a method for determining an interim life safety measure (ILSM) for one or more smoke compartments in the facility.

### BACKGROUND

Generally, to improve healthcare quality, organizations have been developed to inspect and accredit health care facilities. Examples of such organizations include, but are not limited to, centers for Medicare and medic-aid services (CMS) or an accredited independent, not-for-profit organizations, i.e., the joint commission (TJC), det norske veritas (DNV), a center for improvement in healthcare quality (CIHQ), a healthcare facilities accreditation program (HFAP), and the like. For example, the TJC helps in continuously improving health care for the public, by evaluating health care organizations. Further, the TJC accredits and certifies more than 21,000 health care organizations and programs in the United States, including hospitals and health care organizations that provide ambulatory and office-based surgery, behavioral health, home health care, laboratory, and nursing care center services. This organization dominates the market regulating 15,716 healthcare facilities in the United States, 3959 Hospitals, 848 Nursing care Center 363 Critical Access Hospitals 1941 Ambulatory Care 3017 Behavioral Health 5298 Home Care and 290 Office Based Surgery, a total of 78% of the US healthcare facilities.

Further, hospitals may depend to varying degrees on receipt of Federal funds to meet their increasing budget needs. Along with partial Federal financing of hospitals, specific Federal requirements have been introduced requiring hospitals to demonstrate compliance to receive Medicare/Medicaid funding. To demonstrate compliance, hospitals were initially given two options by Congress: Either submit to a federal audit or achieve accreditation from The Joint Commission (originally known as TJC on accreditation of hospitals, and later as the joint commission on accreditation of healthcare organizations). Passing an audit by either of these two organizations would give a hospital “Deemed Status” for a 3-year period which would enable them to collect Medicare/Medicaid funds while they hold this status. Not obtaining or loosing this status would prohibit them from collecting this funding while they were not in “Deemed Status.”

Over the years the healthcare environment has gotten far more complex from a regulatory and technology standpoint as well as a medical and business standpoint. As this has occurred, the number of regulations has increased dramatically; the regulations themselves have become far more complex; documentation requirements have greatly increased; yet due to cost constraints, spending on programs and staffing has been continually cut by the healthcare providers. Currently, hospitals and healthcare facilities lack the ability to conduct risk analysis at the time of the deficiency's discovery, putting pressure on management to conduct risk analysis within a specific mandatory time frame, which may be missed. In addition, incorporating and monitoring multiple interim life safety measures (ILSM) requirements, which are health and safety measures that were put in place to protect the safety of patients, visitors, and staff who work in hospitals and other healthcare facilities, may not happen on time. Since construction, inspection and maintenance activities can impact the facility's life safety; regulations were put in place, which dictate specific mandatory actions, based on risk level, and must be performed with a specific pre-defined timeframe. Facilities must also develop their own ILSM policy for the protection, safety, and health of patients, in order to alleviate hazards, incorporating measures of their own, together with the regulated mandatory requirements.

When under construction or when a failed inspection accrues, a list of Interim Life Safety Measures needs to be determined. The reason for determining and activating the specific ILSM is to protect the safety of patients, visitors, and those who work in the hospital. They are based on conducting risk analysis, to determine which of the ILSM items must be activated and they need to occur within a certain time, from the time of discovery. It is almost impossible for healthcare facility departments to be compliant, at all times, and meet all ILSM compliant requirements, specifically for smoke compartments, which is a type of a passive fire protection within a building and an area within a fire compartment that is required to be separated by barriers (on all sides) such as walls, doors, and/or floors and ceilings having the appropriate resistance to the spread of smoke.

Hence, there is a need to address at least the aforementioned issues/problems in the existing approaches by providing an improved a system and a method for determining an interim life safety measure (ILSM) for one or more smoke compartments in the facility, to ensure the ILSM requirements for a safe environment for all a hospital's occupants are completed, while ensuring and validating that the required activities are performed on time. In addition, the system should ensure a complete documentation audit trail is maintained and all responsible parties are notified, thus providing hospitals with the ability to assure ongoing compliance and reduce cost by replacing their current existing costly manual inspections.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

An aspect of the present disclosure includes a system for determining an interim life safety measure (ILSM) for one or more smoke compartments in a facility. The system analyzes a plurality of potential risk factors associated with one or more individuals in one or more smoke compartments of a facility, based on a type of the one or more smoke compartments. The potential risk factors may a potential harm/hazards to the one or more individuals. The individuals may include, but are not limited to, health care workers, workers, labors, patients, staff, health care providers, vendors, technicians, maintenance staff, visitors, and the like. The type of the one or more smoke compartments may include, but are not limited to, smoke dampers, fire-rated walls, and fire doors smoke barriers, smoke-tight partitions, and the like. Further, the system determines a plurality of risk levels for each of the one or more smoke compartments, based on the analyzed plurality of potential risk factors. Furthermore, the system, in response to determining the plurality of risk levels, classifies one or more assets associated with each of the one or more smoke compartments into the plurality of risk levels, based on at least one of one or more asset classes and a location environment associated with each of the one or more assets in each of the one or more smoke compartments comprising the plurality of risk levels. Additionally, the system assigns a plurality of risk assessment scores to each of one or more inspection points corresponding to the classified one or more assets. The risk assessment score corresponds to at least one of an importance and a potential harm created when the one or more inspection point fails an inspection. Each of the one or more inspection points corresponds to a requirement of an element of performance (EP).

For example, mandated standards include a plurality of elements of performance (EPs) to indicate what needs to be completed to satisfy the standard. An example EP could be “the fire doors need to be inspected once per year.” Embodiments of the present disclosure look not only at what frequency an asset should be inspected, but also how the inspection should be performed (what should be the inspection points). Each inspection point indicates a requirement of the respective EP. For example, an inspection point can be an asset inspection, a document, or an action to be taken. More specifically, an inspection point is any requirement relating to: assets, machinery, instruments and other facility optional equipment, facility condition inspections, construction status and/or inspections relating to having valid current supporting document, as they relate to environmental care, life safety, and emergency management.

Further, the system calculates an aggregated score of the plurality of risk assessment scores associated with a plurality of failed inspection points for the one or more assets within each of the one or more smoke compartments. Furthermore, the system determines one or more ILSMs, when the aggregated score is greater than a pre-determined threshold value for each of the one or more assets within each of the one or more smoke compartments. The ILSM is a health and safety measure to protect the one or more individuals at the facility. Additionally, the system creates at least one of one or more maintenance work orders and one or more corrective work orders, based on the determined one or more ILSMs. Further, the system outputs, to user device associated with a user, one or more notifications corresponding to a status of the created at least one of the one or more maintenance work orders and the one or more corrective work orders. The one or more notifications inform the user to provide instructions for creating the at least one of the one or more maintenance work orders or the one or more corrective work orders and inform the user to monitor the status.

Another aspect of the present disclosure includes a method for determining an interim life safety measure (ILSM) for one or more smoke compartments in a facility. The method includes analyzing a plurality of potential risk factors associated with one or more individuals in one or more smoke compartments of a facility, based on a type of the one or more smoke compartments. The individuals may include, but are not limited to, health care workers, workers, labors, patients, staff, health care providers, vendors, technicians, maintenance staff, visitors, and the like. Further, the method includes determining a plurality of risk levels for each of the one or more smoke compartments, based on the analyzed plurality of potential risk factors. Furthermore, the method includes, in response to determining the plurality of risk levels, classifying one or more assets associated with each of the one or more smoke compartments into the plurality of risk levels, based on at least one of one or more asset classes and a location environment associated with each of the one or more assets in each of the one or more smoke compartments comprising the plurality of risk levels. Additionally, the method includes assigning a plurality of risk assessment scores to each of one or more inspection points corresponding to the classified one or more assets. The risk assessment score corresponds to at least one of an importance and a potential harm created when the one or more inspection point fails an inspection. Each of the one or more inspection points corresponds to a requirement of an element of performance (EP).

Further, the method includes calculating an aggregated score of the plurality of risk assessment scores associated with a plurality of failed inspection points for the one or more assets within each of the one or more smoke compartments. Furthermore, the method includes determining one or more ILSMs, when the aggregated score is greater than a pre-determined threshold value for each of the one or more assets within each of the one or more smoke compartments. The ILSM is a health and safety measure to protect the one or more individuals at the facility. Additionally, the method includes creating at least one of one or more maintenance work orders and one or more corrective work orders, based on the determined one or more ILSMs. Further, the method includes outputting, to a user device associated with user, one or more notifications corresponding to a status of the created at least one of the one or more maintenance work orders and the one or more corrective work orders. The one or more notifications inform the user to provide instructions for creating the at least one of the one or more maintenance work orders or the one or more corrective work orders and inform the user to monitor the status.

Another aspect of the present disclosure includes a non-transitory computer-readable medium comprising machine-readable instructions that are executable by a hardware processor to analyze a plurality of potential risk factors associated with one or more individuals in one or more smoke compartments of a facility, based on a type of the one or more smoke compartments. Further, the processor determines a plurality of risk levels for each of the one or more smoke compartments, based on the analyzed plurality of potential risk factors. Furthermore, the processor, in response to determining the plurality of risk levels, classifies one or more assets associated with each of the one or more smoke compartments into the plurality of risk levels, based on at least one of one or more asset classes and a location environment associated with each of the one or more assets in each of the one or more smoke compartments comprising the plurality of risk levels. Additionally, the processor assigns a plurality of risk assessment scores to each of one or more inspection points corresponding to the classified one or more assets. The risk assessment score corresponds to at least one of an importance and a potential harm created when the one or more inspection point fails an inspection. Each of the one or more inspection points corresponds to a requirement of an element of performance (EP).

Further, the processor calculates an aggregated score of the plurality of risk assessment scores associated with a plurality of failed inspection points for the one or more assets within each of the one or more smoke compartments. Each inspection point indicates a requirement of an element of performance (EP). During inspections, a sum of all risk assessment scores (aggregated score) associated with the failed inspection points within the smoke compartment is calculated. Furthermore, the processor determines one or more Interim Life Safety Measure (ILSM), when the aggregated score is greater than a pre-determined threshold value for each of the one or more assets within each of the one or more smoke compartments. The ILSM is a health and safety measure to protect the one or more individuals at the facility. Additionally, the processor creates at least one of one or more maintenance work orders and one or more corrective work orders, based on the determined one or more ILSMs. Further, the processor outputs, to a user device associated with a user, one or more notifications corresponding to a status of the created at least one of the one or more maintenance work orders and the one or more corrective work orders. The one or more notifications inform the user to provide instructions for creating the at least one of the one or more maintenance work orders or the one or more corrective work orders and informs the user to monitor the status.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates an exemplary block diagram representation of a network architecture for a system for determining an interim life safety measure (ILSM) for one or more smoke compartments in a facility, in accordance with an embodiment of the present disclosure;

FIG. 2 illustrates an exemplary block diagram representation of a detailed view of the system, in accordance with an embodiment of the present disclosure;

FIG. 3 illustrates an exemplary block diagram representation of an enterprise mobile, cloud-based compliance system to increase hospital facility management compliance and to improve management and maintenance of healthcare facilities, in accordance with an embodiment of the present disclosure;

FIG. 4 illustrates an exemplary flow diagram representation of an inspection method for compliance and risk assessment, in accordance with an embodiment of the present disclosure;

FIG. 5 illustrates an exemplary block diagram representation of elements of a compliance and risk assessment computer program, in accordance with an embodiment of the present disclosure;

FIG. 6 illustrates an exemplary flow diagram representation of a process for risk assessment in a compliance system, in accordance with an embodiment of the present disclosure;

FIG. 7 illustrates an exemplary flow diagram representation of a method for issuing an ILSM, in accordance with an embodiment of the present disclosure;

FIG. 8 illustrates a flow chart depicting a method for determining an interim life safety measure (ILSM) for one or more smoke compartments in a facility, according to an example embodiment of the present disclosure; and

FIG. 9 illustrates an exemplary block diagram representation of a hardware platform for implementation of the disclosed system, according to an example embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated online platform, and such further applications of the principles of the disclosure as would normally occur to those skilled in the art are to be construed as being within the scope of the present disclosure.

For simplicity and illustrative purposes, the present disclosure is described by referring mainly to examples thereof. The examples of the present disclosure described herein may be used together in different combinations. In the following description, details are set forth in order to provide an understanding of the present disclosure. It will be readily apparent, however, that the present disclosure may be practiced without limitation to all these details. Also, throughout the present disclosure, the terms “a” and “an” are intended to denote at least one example of a particular element. The terms “a” and “an” may also denote more than one example of a particular element. In the following specification and the claims, reference will be made to a number of terms, which shall be defined to have the following meanings. The singular forms “a,” “an,” and “the” include plural references unless the context clearly dictates otherwise.

As used herein, the term “includes” means includes but not limited to, the term “including” means including but not limited to. The term “based on” means based at least in part on, the term “based upon” means based at least in part upon, and the term “such as” means such as but not limited to. The term “relevant” means closely connected or appropriate to what is being performed or considered. The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or subsystems or elements, structures, or components preceded by “comprises . . . a” does not, without more constraints, preclude the existence of other devices, subsystems, elements, structures, components, additional devices, additional subsystems, additional elements, additional structures, or additional components. Appearances of the phrase “in an embodiment”, “in another embodiment” and similar language throughout this specification may, but not necessarily do, all refer to the same embodiment.

Before describing in detail exemplary embodiments, it is noted that the embodiments reside primarily in combinations of components and procedures related to the apparatus. Accordingly, the apparatus components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

Unless otherwise defined, all technical and scientific terms used herein have the same meaning as commonly understood by those skilled in the art to which this disclosure belongs. The system, methods, and examples provided herein are only illustrative and not intended to be limiting.

Various embodiments of the present disclosure provide a system and a method for determining an interim life safety measure (ILSM) for one or more smoke compartments in a facility. The smoke compartments are designated areas within a building that are separated from each other by fire-resistant barriers, such as walls, doors, floors, or ceilings, and the like, to prevent the spread of smoke and fire. The purpose of smoke compartments is to provide a safe means of egress for occupants of a building during a fire. In the event of a fire, smoke compartments help to contain smoke and heat, which can make it easier for firefighters to locate and extinguish the fire. They also allow for a safer evacuation of building occupants by preventing the spread of smoke and fire to other areas of the building. The smoke compartments are commonly found in high-rise buildings, hospitals, hotels, and other large structures. They are typically identified by signs or labels and are often included in building codes and fire safety regulations.

The present disclosure provides a system and method that affords hospitals the ability to assure ongoing compliance and reduce cost by migrating from their current existing costly manual inspections to using a central repository and tracking solution that helps improve healthcare physical environment quality of service and provides for successful compliance outcomes. Embodiments of the present disclosure ensure Interim Life Safety Measures (ILSMs) are completed, while also ensuring and validating the required activities are performed on time. Thus, embodiments of the present disclosure schedule regulatory compliance activities around regulatory mandated timelines. Embodiments of the present disclosure also provide warnings and alarms when deadlines approach, give emergency notifications to users when deadlines have not been met, and provide ongoing, comprehensive regulatory compliance reports to assist in the management of these programs. As a result, hospitals can begin a survey knowing required compliance activities have been completed on time and documented correctly, thus dramatically reducing the probability of being cited overall.

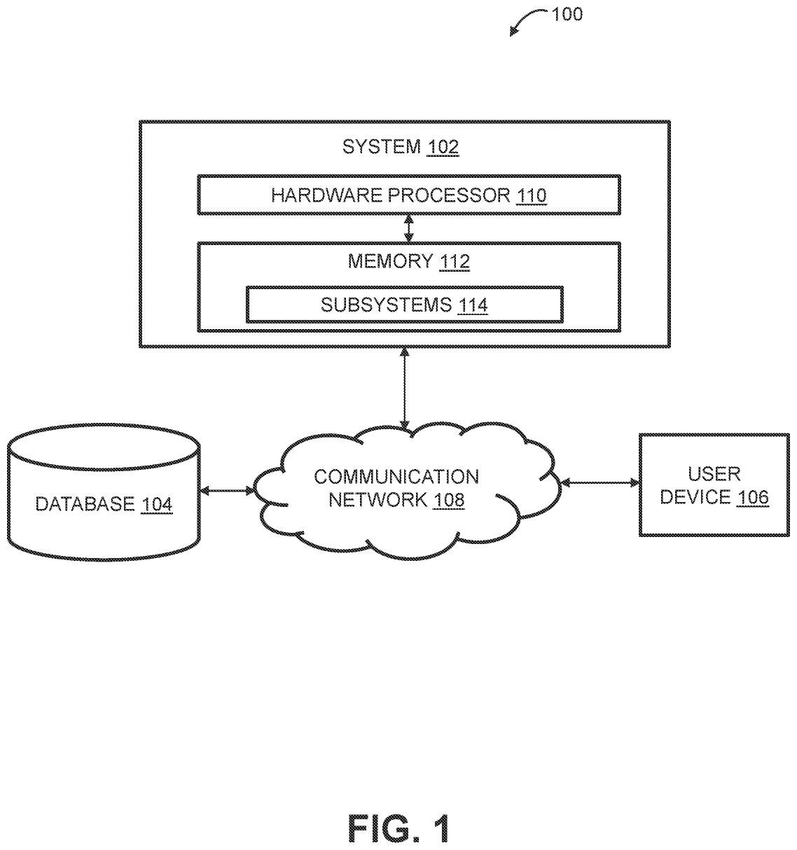

FIG. 1 illustrates an exemplary block diagram representation of a network architecture 100 for a system 102 for determining an interim life safety measure (ILSM) for one or more smoke compartments in a facility, in accordance with an embodiment of the present disclosure. The network architecture 100 may include the system 102, a database 104, and a user device 106. The system 102 may be communicatively coupled to the database 104, and the user device 106 via a communication network 108. The communication network 108 may be a wired communication network and/or a wireless communication network. The database 104 may include, but is not limited to, compartment data of a plurality of compartments for a facility, wherein each compartment defines a portion of the facility, risk assessment score for each inspection point, wherein the risk assessment score is a number indicating an importance and potential harm created when the inspection point fails inspection, a list of ILSM actions, correlating relationship data for correlating relationships between the types of deficiencies that may be encountered and the ILSM actions that address these deficiencies, any other content, and combinations thereof.

Further, the user device 106 may be associated with, but not limited to, a user, an administrator, a vendor, a technician, health care worker, a supervisor, compliance team, an entity, a facility, and the like. The user device 106 may be used to provide input and/or receive output to/from the system 102. The user device 106 may present to the user one or more user interfaces for the user to interact with the system 102 for the interim life safety measure (ILSM) needs.

The user device 106 may be at least one of, an electrical, an electronic, an electromechanical, and a computing device. The user device 106 may include, but is not limited to, a mobile device, a smart phone, a Personal Digital Assistant (PDA), a tablet computer, a phablet computer, a wearable computing device, a Virtual Reality/Augmented Reality (VR/AR) device, a laptop, a desktop, a server, and the like. The entities and the facility may include, but are not limited to, a hospital, an e-commerce company, a merchant organization, an airline company, a hotel booking company, a company, an outlet, a manufacturing unit, an enterprise, an organization, an educational institution, a secured facility, a warehouse facility, a supply chain facility, any other facility and the like.

Further, the system 102 may be implemented by way of a single device or a combination of multiple devices that may be operatively connected or networked together. The system 102 may be implemented in hardware or a suitable combination of hardware and software. The system 102 includes a hardware processor 110 and a memory 112. The memory 112 may include a plurality of subsystems 114.

The system 102 may be a hardware device including the hardware processor 110 executing machine-readable program instructions for determining an interim life safety measure (ILSM) for one or more smoke compartments in a facility. The one or more smoke compartments includes, but are not limited to, high-rise buildings, hospitals, hotels, office buildings, residential buildings, and the like. The high-rise building are the buildings that are taller than six stories usually require smoke compartments to provide a safe means of egress during a fire. Further, the smoke compartments in hospitals are used to separate patient care areas from other areas of the building, such as administrative offices, to prevent the spread of smoke and fire. Furthermore, large hotels often have smoke compartments to separate guest rooms from other areas of the building, such as lobbies and restaurants. The smoke compartments can be used in office buildings to separate different departments or floors from each other, which can help prevent the spread of smoke and fire. The smoke compartments can be used in apartment buildings to separate individual units from each other, which can help prevent the spread of smoke and fire. Any building that requires multiple areas to be separated from each other to prevent the spread of smoke and fire may have smoke compartments.

Execution of the machine-readable program instructions by the hardware processor 110 may enable the proposed system 102 to determine an interim life safety measure (ILSM) for one or more smoke compartments in a facility. The “hardware” may comprise a combination of discrete components, an integrated circuit, an application-specific integrated circuit, a field-programmable gate array, a digital signal processor, or other suitable hardware. The “software” may comprise one or more objects, agents, threads, lines of code, subroutines, separate software applications, two or more lines of code, or other suitable software structures operating in one or more software applications or on one or more processors.

The hardware processor 110 may include, for example, microprocessors, microcomputers, microcontrollers, digital signal processors, central processing units, state machines, logic circuits, and/or any devices that manipulate data or signals based on operational instructions. Among other capabilities, hardware processor 110 may fetch and execute computer-readable instructions in a memory operationally coupled with the system 102 for performing tasks such as data processing, input/output processing, and/or any other functions. Any reference to a task in the present disclosure may refer to an operation being or that may be performed on data.

Though few components and subsystems are disclosed in FIG. 1, there may be additional components and subsystems which is not shown, such as, but not limited to, assets, machinery, instruments, facility equipment, optional equipment, facility condition inspection equipment, heating, ventilation and air-conditioning systems (HVAC), environmental care devices, life safety devices, intensive care devices, treatment devices, emergency management devices, health care device, door closures, and the like. The person skilled in the art should not be limiting the components/subsystems shown in FIG. 1.

In an exemplary embodiment, the system 102 may execute the hardware processor 110 to analyze a plurality of potential risk factors associated with one or more individuals in one or more smoke compartments of a facility, based on a type of the one or more smoke compartments. In an exemplary embodiment, the plurality of risk levels includes one or more dynamic values for each of the one or more asset classes, based on the location environment associated with each of the one or more assets. The individuals may include, but are not limited to, health care workers, workers, labors, patients, staff, health care providers, vendors, technicians, maintenance staff, visitors, and the like. For example, the one or more smoke compartments are designed to limit the spread of smoke within a building and protect occupants from the harmful effects of smoke inhalation. However, there are plurality of potential risk factors to one or more individuals in the one or more smoke compartments that can compromise their safety. Examples of potential risk factors to one or more individuals include, but are not limited to, lack of oxygen, toxic smoke, physical obstructions, panic, injuries, and the like. Lack of oxygen: the smoke compartments are designed to contain smoke, but they can also contain oxygen. If the smoke compartment is not properly ventilated, occupants may experience a lack of oxygen, which can lead to dizziness, confusion, and unconsciousness. Toxic smoke: Smoke from a fire can be toxic, containing carbon monoxide, hydrogen cyanide, and other dangerous chemicals. If occupants are exposed to toxic smoke for an extended period, it can cause serious health problems and even death. Physical obstructions: the smoke compartments may be obstructed by debris or furniture, preventing occupants from exiting the compartment safely. Panic: In a fire emergency, occupants may panic, leading to disorientation and difficulty in finding the exits. Panic can also lead to poor decision-making and reckless behavior, putting occupants at greater risk. Injuries: Smoke compartments may contain hazards such as sharp objects, hot surfaces, or tripping hazards that can cause injuries to occupants.

Further, the risk levels include low risk, medium risk, high risk and highest risk level. The low risk level includes non-patient care areas, office areas, plant area. Further, the medium risk level includes patient care support areas such as cardiology, echocardiography, endoscopy, fitness center, kitchen/cafeteria, lobby, nuclear medicine, outpatient area, radiology (all excluding magnetic resonance imaging (MRI)), radiology (MRI only), respiratory therapy rooms, and the like. Furthermore, the high-risk level includes patient care areas such as emergency room, inpatient unit, intensive care units, labor & delivery, laboratories (specimen), medical units, negative pressure isolation rooms, newborn nursery, oncology, operating rooms including c-section, orthopedics, outpatient surgery, physical therapy, and the like. Furthermore, the highest risk level includes procedural, invasive, sterile support and highly compromised patient care areas such as any area caring for immunocompromised patients, burn unit cardiac cath/electrophysiology lab, central sterile supply, critical care unit, pediatrics, pharmacy (incl retail pharmacy), post-anesthesia care unit, surgical units, and the like.

In an exemplary embodiment, the system 102 may execute the hardware processor 110 to determine a plurality of risk levels for each of the one or more smoke compartments, based on the analyzed plurality of potential risk factors.

In an exemplary embodiment, the system 102 may execute the hardware processor 110 to, in response to determining the plurality of risk levels, classify one or more assets associated with each of the one or more smoke compartments into the plurality of risk levels, based on at least one of one or more asset classes and a location environment associated with each of the one or more assets in each of the one or more smoke compartments comprising the plurality of risk levels.

In an exemplary embodiment, the system 102 may execute the hardware processor 110 to assign a plurality of risk assessment scores to each of one or more inspection points corresponding to the classified one or more assets. In an exemplary embodiment, the risk assessment score corresponds to at least one of an importance and a potential harm created when the one or more inspection point fails an inspection. Each of the one or more inspection points corresponds to a requirement of an element of performance (EP). In an exemplary embodiment, the risk assessment is not performed for a failed inspection point when the risk assessment score associated with the failed inspection point is zero. In an exemplary embodiment, the risk assessment for a failed inspection point is delayed for a predetermined time when a “time to resolve” option is selected for a failed inspection point, and wherein risk assessment for the failed inspection point is performed when the failed inspection point is not completed within the predefined time.

In an exemplary embodiment, the system 102 may execute the hardware processor 110 to calculate an aggregated score of the plurality of risk assessment scores associated with a plurality of failed inspection points for the one or more assets within each of the one or more smoke compartments.

In an exemplary embodiment, the system 102 may execute the hardware processor 110 to determine one or more ILSMs, when the aggregated score is greater than a pre-determined threshold value for each of the one or more assets within each of the one or more smoke compartments. The ILSM is a health and safety measure to protect the one or more individuals at the facility.

In an exemplary embodiment, the system 102 may execute the hardware processor 110 to create at least one of one or more maintenance work orders and one or more corrective work orders, based on the determined one or more ILSMs.

In an exemplary embodiment, the system 102 may execute the hardware processor 110 to output, to the user device 106 associated with the user, one or more notifications corresponding to a status of the created at least one of the one or more maintenance work orders and the one or more corrective work orders. The one or more notifications informs the user to provide instructions for creating the at least one of the one or more maintenance work orders or the one or more corrective work orders and informs the user to monitor the status. In an exemplary embodiment, the system 102 may execute the hardware processor 110 to generate the one or more notifications that the determined ILSM is no longer needed when at least one of the one or more work orders and the one or more corrective work orders associated with the generated ILSM are completed.

In an exemplary embodiment, the system 102 may execute the hardware processor 110 to determine the status of at least one of the one or more maintenance work orders is indicated as not completed and the status of the one or more corrective work orders comprises one or more reported deficiencies requiring an immediate corrective action.

In an exemplary embodiment, the system 102 may execute the hardware processor 110 to retrieve from the database 104, a risk profile corresponding to the location environment associated with each of the one or more assets in each of the one or more smoke compartments. In an exemplary embodiment, the risk profile includes, but not limited to, the potential risk factors, the plurality of risk levels, plurality of risk assessment scores, the aggregated score, and the like.

In an exemplary embodiment, the system 102 may execute the hardware processor 110 to prioritize each of at least of the one or more maintenance work orders and the one or more corrective work orders, based on the risk profile.

In an exemplary embodiment, the system 102 may execute the hardware processor 110 to determine, when the failed inspection points correspond to risk assessment assets, the location environment of the one or more assets corresponding to at least one of one or more supplementary facilities and one or more third party vendors, and a proximity to a subsequently discovered risk assessment assets.

In an exemplary embodiment, the system 102 may execute the hardware processor 110 to increment the risk assessment score, in response to the determined risk assessment assets impacting the aggregated risk assessment score.

In an exemplary embodiment, the system 102 may execute the hardware processor 110 to store in the database 104, a plurality of ILSM actions and a plurality of correlating deficiency assets. In an exemplary embodiment, each ILSM action defines an action related to protecting the individuals at the facility. Further, each correlating deficiency asset defines a correlation between a deficiency encountered at the facility and at least one ILSM action. In an exemplary embodiment, the ILSM is determined by generating a list of the selected ILSM actions. In an exemplary embodiment, the system 102 may execute the hardware processor 110 to select a plurality of the plurality of ILSM actions based on deficiencies related to the failed inspection points and related correlating deficiency items.

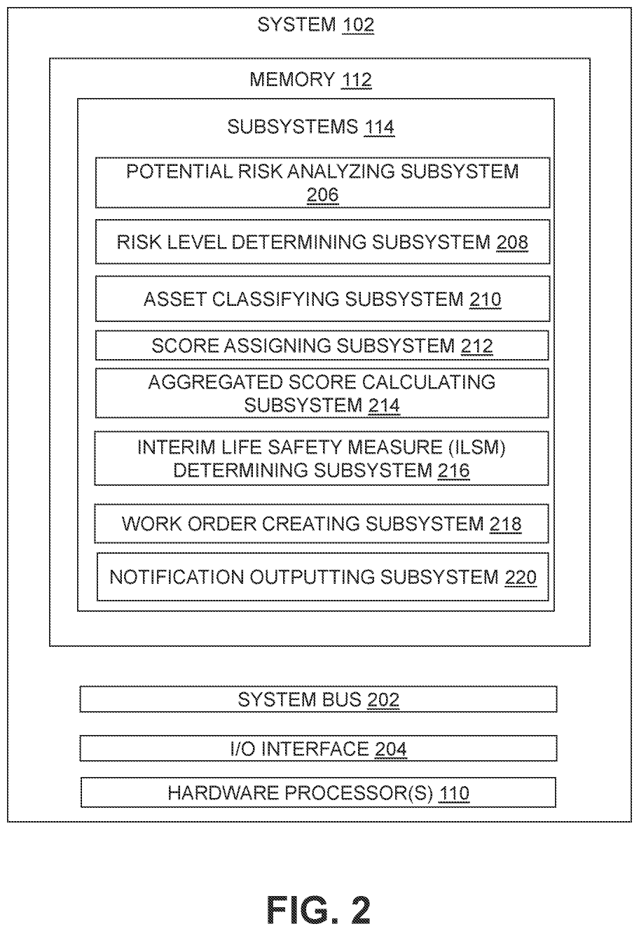

FIG. 2 illustrates an exemplary block diagram representation of a detailed view of the system 102, in accordance with an embodiment of the present disclosure. The system 102 includes the hardware processor 110. The system 102 also includes a memory 112 coupled to the hardware processor 110. The memory 112 includes a set of program instructions in the form of the plurality of subsystems 114.

The hardware processor(s) 110, as used herein, means any type of computational circuit, such as, but not limited to, a microprocessor, a microcontroller, a complex instruction set computing microprocessor, a reduced instruction set computing microprocessor, a very long instruction word microprocessor, an explicitly parallel instruction computing microprocessor, a digital signal processor, or any other type of processing circuit, or a combination thereof.

The memory 112 includes the plurality of subsystems 104 stored in the form of executable program which instructs the hardware processor 110 via a system bus 202 to perform the above-mentioned method steps. Further, the system 102 may include an Input/Output (I/O) interface 204, which may be used to receive user inputs, from the user devices 106 associated with the users (not shown), and store/retrieve data from the database 104.

Further, the system 102 includes a memory 112 to store executable program and the plurality of subsystems 114. The plurality of subsystems 114 include a potential risk analyzing subsystem 206, a risk level determining subsystem 208, an asset classifying subsystem 210, a score assigning subsystem 212, an aggregated score calculating subsystem 214, an Interim Life Safety Measure (ILSM) determining subsystem 216, a work order creating subsystem 218, and a notification outputting subsystem 220.

The plurality of subsystems 114 may be stored within the memory 112. In an example, the plurality of subsystems 114 communicatively coupled to the hardware processor 110 configured in the system 102, may also be present outside the memory 112, and implemented as hardware. As used herein, the term “subsystems” may refer to an Application-Specific Integrated Circuit (ASIC), an electronic circuit, a processor (shared, dedicated, or group) and memory that execute one or more software or firmware programs, a combinational logic circuit, and/or other suitable components that provide the described functionality.

Further, the system 102 may also include other units such as a display unit, an input unit, an output unit, and the like, however the same are not shown in FIG. 1 and FIG. 2, for the purpose of clarity. Also, in FIG. 2 only a few units are shown, however, the system 102 or the network architecture 100 may include multiple such units or the system 102/network architecture 100 may include any such numbers of the units, obvious to a person skilled in the art or as required to implement the features of the present disclosure.

Computer memory elements may include any suitable memory device(s) for storing data and executable program, such as read only memory, random access memory, erasable programmable read only memory, electronically erasable programmable read only memory, hard drive, removable media drive for handling memory cards and the like. Embodiments of the present subject matter may be implemented in conjunction with program modules, including functions, procedures, data structures, and application programs, for performing tasks, or defining abstract data types or low-level hardware contexts. Executable program stored on any of the above-mentioned storage media may be executable by the hardware processor(s) 110.

In an exemplary embodiment, the system 102 may execute the potential risk analyzing subsystem 206 to analyze a plurality of potential risk factors associated with one or more individuals in one or more smoke compartments of a facility, based on a type of the one or more smoke compartments. In an exemplary embodiment, the plurality of potential risk factors may be analyzed using at least one of, but not limited to, an artificial intelligence (AI) based smoke detectors, a predictive maintenance based AI model, Real-time monitoring based AI model, emergency response based AI model, occupant tracking based AI model, and the like.

For example, smoke detection systems: AI-powered smoke detectors can be trained to differentiate between different types of smoke and alert building managers to potential fire risks. These systems can use machine learning algorithms to analyze data on the composition and behavior of smoke and identify patterns that indicate a potential fire. In predictive maintenance, the AI can be used to analyze data on the condition and performance of building systems such as HVAC and electrical systems. By identifying patterns of failure and predicting equipment malfunctions, AI can help building managers schedule preventive maintenance and reduce the risk of equipment failure and fire. Furthermore, for real-time monitoring, the AI-powered sensors can be installed throughout a building to monitor temperature, humidity, and other environmental factors. These sensors can be linked to a central AI system that can analyze the data in real-time and provide alerts to building managers when conditions are conducive to a fire. Additionally, using emergency response systems, the AI can be used to coordinate emergency response efforts in the event of a fire. For example, AI-powered systems can analyze data on the location of the fire, the number of occupants in the building, and the location of emergency responders to provide real-time information to emergency responders and help them respond quickly and efficiently. Further, for occupant tracking, the AI-powered systems can track the location of occupants in a building and provide real-time information on their movements. In the event of a fire, this information can be used to locate and evacuate occupants quickly and safely.

Examples of AI based models for analyzing potential risk factors to one or more individuals in smoke compartments include, but are not limited to, convolutional neural networks (CNNs), recurrent neural networks (RNNs), Bayesian networks, support vector machines (SVMs), decision trees, and the like. The CNNs are a type of deep learning model that can analyze images and video data to detect smoke or flames in a smoke compartment. These models are trained on large datasets of smoke and non-smoke images to learn to accurately classify smoke and alert building managers to potential fire risks. The RNNs are a type of deep learning model that can analyze data over time and make predictions about future events. These models can be used to analyze data from sensors in a smoke compartment to identify patterns that may indicate a fire risk. For example, an RNN model could analyze data from temperature or humidity sensors to predict when conditions in a smoke compartment are conducive to a fire. The Bayesian Networks are a probabilistic graphical model that can be used to model the causal relationships between different factors that contribute to a fire risk. For example, a Bayesian Network could model the relationships between factors such as the age of building systems, the presence of flammable materials, and the location of smoke detectors to identify areas of high fire risk. The SVMs are a type of machine learning model that can analyze data to identify patterns and classify data into different categories. SVMs can be used to analyze data from sensors in a smoke compartment to identify patterns that may indicate a fire risk, such as changes in temperature, humidity, or smoke density. Decision trees are a type of machine learning model that can analyze data and make predictions based on a series of binary decisions. These models can be used to analyze data from sensors in a smoke compartment to identify potential fire risks and provide recommendations for preventive measures or emergency response.

In an exemplary embodiment, the plurality of risk levels includes one or more dynamic values for each of the one or more asset classes, based on the location environment associated with each of the one or more assets. For example, one or more asset classes in hospital smoke compartments can include various types of equipment and furnishings that are required for patient care and safety, heating, ventilation and air-conditioning systems (HVAC) system, fire systems (alarms, sprinklers), electrical system, fire doors, other mechanical systems present in the room.

For instance, medical equipment, such as patient monitors, infusion pumps, ventilators, and dialysis machines, are essential for patient care and can be considered an asset class in hospital smoke compartments. The furniture and fixtures such as Hospital beds, exam tables, chairs, and other furnishings used in patient rooms and treatment areas are also considered an asset class within smoke compartments. An emergency equipment, such as fire extinguishers, fire alarms, and emergency lighting, are critical components of hospital smoke compartments and are necessary to ensure the safety of patients, staff, and visitors. The building infrastructure, including heating, ventilation and air-conditioning systems (HVAC) systems, electrical systems, and plumbing systems, can also be considered an asset class within smoke compartments, as these systems must be designed and maintained to prevent the spread of smoke and fire. A personal protective equipment (PPE) such as masks, gloves, and gowns, are essential for preventing the spread of infectious diseases in hospitals and can also be considered an asset class within smoke compartments.

For example, the plurality of risk levels for the one or more assets may be based on inheritance or acquisition. The inheritance of the plurality of risks/risk levels are based on the one or more asset classes. Further, acquisition of the risks/risk levels is based on the location environment of the asset classes. The plurality of risk levels are not a static value for an asset type, it can be higher or lower for same asset, based on the asset location. For instance, a heating, ventilation and air-conditioning systems (HVAC) unit in a negative pressure room may include risk score=very high while the risk level for the exact same asset when the HVAC location is in a cafeteria or lobby is a score=Low. When an inspection of an asset fails, the system 102 may consider addition risk assessment, a risk profile based on the location environment of the assets. The risk profile includes different risks for different environments, for example, negative-air pressure rooms, emergency rooms (ER), and the like. The risk profile may be generated based on the type of asset/asset classes and possibility of harm/potential risk factors, based on location of assets, amount of risk in areas such as lobby vs. emergency rooms, different departments such as infectious diseases vs. emergency rooms vs. cafeteria, and the like.

Further, the inheritance or the acquisition for the asset class may be based on using inheritance or acquisition-based AI models. Such AI models may include convolutional neural networks (CNNs), random forests, SVM, long short-term memory (LSTM) networks, generative adversarial networks (GANs), and the like. For example, the CNNs are a type of deep learning model that can analyze images and classify them based on their visual features. For example, a CNN could be trained to classify real estate properties based on their architectural style, location, and age. The random forests are a type of decision tree model that can be used to classify data into multiple classes based on a combination of features. For example, a Random Forest model could be trained to classify stocks into different asset classes based on their historical performance, industry sector, and financial metrics. The SVMs are a type of machine learning model that can be used to classify data into different categories based on a set of features. For example, an SVM model could be trained to classify artworks into different asset classes based on their style, medium, and historical auction prices. The LSTMs are a type of recurrent neural network that can be used to analyze time-series data and make predictions about future values. For example, an LSTM model could be trained to predict the asset class of a stock based on its historical performance over time. The GANs are a type of deep learning model that can be used to generate new data samples that are similar to a given dataset. For example, a GAN model could be trained to generate new artworks that are similar in style to existing artworks in a particular asset class.

In an example, certain areas/location environment such as immunocompromised patients, surgery, intensive care, neonatal intensive care unit (NICU) may be assigned as very high-risk level. Further, certain areas/location environment such as front lobby waiting area or cafeteria may be assigned as low risk level.

In an exemplary embodiment, the system 102 may execute the risk level determining subsystem 208 to determine a plurality of risk levels for each of the one or more smoke compartments, based on the analyzed plurality of potential risk factors.

In an exemplary embodiment, the system 102 may execute the asset classifying subsystem 210 to, in response to determining the plurality of risk levels, classify one or more assets associated with each of the one or more smoke compartments into the plurality of risk levels, based on at least one of one or more asset classes and a location environment associated with each of the one or more assets in each of the one or more smoke compartments comprising the plurality of risk levels.

In an exemplary embodiment, the system 102 may execute the score assigning subsystem 212 to assign a plurality of risk assessment scores to each of one or more inspection points corresponding to the classified one or more assets. In an exemplary embodiment, the risk assessment score corresponds to at least one of an importance and a potential harm created when the one or more inspection point fails an inspection. Each of the one or more inspection points corresponds to a requirement of an element of performance (EP). In an exemplary embodiment, the risk assessment is not performed for a failed inspection point when the risk assessment score associated with the failed inspection point is zero. In an exemplary embodiment, the risk assessment for a failed inspection point is delayed for a predetermined time when a “time to resolve” option is selected for a failed inspection point, and wherein risk assessment for the failed inspection point is performed when the failed inspection point is not completed within the predefined time.

In an exemplary embodiment, the system 102 may execute the aggregated score calculating subsystem 214 to calculate an aggregated score of the plurality of risk assessment scores associated with a plurality of failed inspection points for the one or more assets within each of the one or more smoke compartments.

In an exemplary embodiment, the system 102 may execute the Interim Life Safety Measure (ILSM) determining subsystem 216 to determine one or more ILSMs, when the aggregated score is greater than a pre-determined threshold value for each of the one or more assets within each of the one or more smoke compartments. The ILSM is a health and safety measure to protect the one or more individuals at the facility.

In an exemplary embodiment, the system 102 may execute the work order creating subsystem 218 to create at least one of one or more maintenance work orders and one or more corrective work orders, based on the determined one or more ILSMs.

In an exemplary embodiment, the system 102 may execute the notification outputting subsystem 220 to output, to the user device 106 associated with a user, one or more notifications corresponding to a status of the created at least one of the one or more maintenance work orders and the one or more corrective work orders. The one or more notifications inform the user to provide instructions for creating the at least one of the one or more maintenance work orders or the one or more corrective work orders and inform the user to monitor the status. In an exemplary embodiment, the system 102 may execute the notification outputting subsystem 220 to generate the one or more notifications that the determined ILSM is no longer needed when at least one of the one or more work orders and the one or more corrective work orders associated with the generated ILSM are completed.

In an exemplary embodiment, the system 102 may execute the work order creating subsystem 218 to determine the status of at least one of the one or more maintenance work orders is indicated as not completed and the status of the one or more corrective work orders comprises one or more reported deficiencies requiring an immediate corrective action.

In an exemplary embodiment, the system 102 may execute a profile retrieving subsystem (not shown) to retrieve from the database 104, a risk profile corresponding to the location environment associated with each of the one or more assets in each of the one or more smoke compartments. In an exemplary embodiment, the risk profile includes, but not limited to, the potential risk factors, the plurality of risk levels, plurality of risk assessment scores, the aggregated score, and the like.

In an exemplary embodiment, the system 102 may execute the work order creating subsystem 218 to prioritize each of at least of the one or more maintenance work orders and the one or more corrective work orders, based on the risk profile.

In an exemplary embodiment, the system 102 may execute a failed inspection determining subsystem (not shown) to determine, when the failed inspection points correspond to risk assessment assets, the location environment of the one or more assets corresponding to at least one of one or more supplementary facilities and one or more third party vendors, and a proximity to a subsequently discovered risk assessment assets.

In an exemplary embodiment, the system 102 may execute the score assigning subsystem 212 to increment the risk assessment score, in response to the determined risk assessment assets impacting the aggregated risk assessment score.

In an exemplary embodiment, the system 102 may execute an action storing subsystem (not shown) to store in the database 104, a plurality of ILSM actions and a plurality of correlating deficiency assets. In an exemplary embodiment, each ILSM action defines an action related to protecting the individuals at the facility. Further, each correlating deficiency asset defines a correlation between a deficiency encountered at the facility and at least one ILSM action. In an exemplary embodiment, the ILSM is determined by generating a list of the selected ILSM actions. In an exemplary embodiment, the system 102 may execute an action selecting subsystem (not shown) to select a plurality of the plurality of ILSM actions based on deficiencies related to the failed inspection points and related correlating deficiency items.

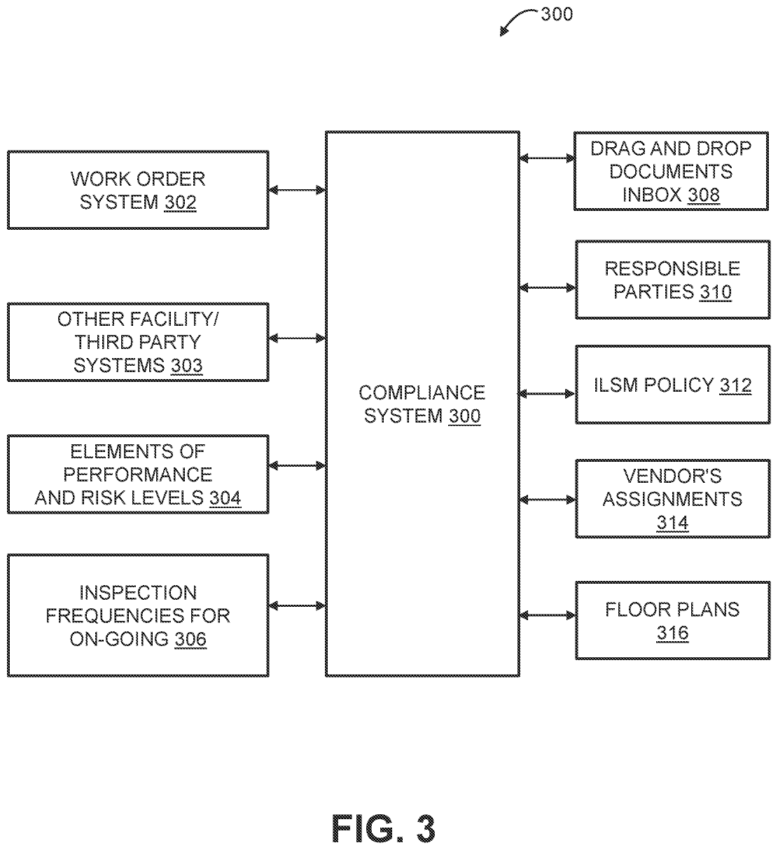

FIG. 3 illustrates an exemplary block diagram representation of an enterprise mobile, a cloud-based compliance system 300 (i.e., system 102) to increase hospital facility management compliance and to improve management and maintenance of healthcare facilities, in accordance with an embodiment of the present disclosure. Embodiments of the present disclosure integrate with multiple sources of existing data to determine where the facility is in terms of compliance, what needs to be performed, and provide the facility a schedule of what needs to be completed. To perform these functions, the compliance system 300 (i.e., system 102) is initially set up to operate in a specific environment as discussed below. The smoke compartments in hospitals include, but are not limited to, patient care areas, operating rooms, operating rooms, emergency department, intensive care unit (ICU), imaging and diagnostic areas, imaging and diagnostic areas, and the like.

The patient care areas in hospitals typically have multiple smoke compartments to separate patient care areas, such as hospital rooms, nursing stations, and treatment rooms, from other areas of the building, such as administrative offices, lobbies, and mechanical rooms. Further, operating rooms are often located within a smoke compartment that is separated from other areas of the hospital to prevent the spread of smoke and fire. Furthermore, emergency department of a hospital may have a smoke compartment to separate the treatment area from other parts of the building, such as waiting rooms and administrative offices. Additionally, the intensive care unit (ICU) may be often located within a smoke compartment to protect critically ill patients from smoke and fire. The imaging and diagnostic areas, such as radiology and magnetic resonance imaging (MRI) rooms, may be located within a smoke compartment to prevent the spread of smoke and fire to other areas of the hospital. Smoke compartments in hospitals are designed to protect patients, staff, and visitors from the dangers of smoke and fire, and are typically required by building codes and fire safety regulations.

The set up to operate in a specific environment includes integrating the compliance and risk assessment system 300 with a work order system 302 associated with the facility, other facility/third party systems 303, providing elements of performance (EP) 304 and associated risk assessment scores based on mandated frequencies and risk levels, providing on-going inspection frequencies 306 associated with the facility such as a hospital, and setting up an inbox 308 for documents emailed to the compliance system 300. In addition, responsible parties 310 are associated with each EP, an interim life safety measures policy 312 is provided to the compliance system 300, as are vendor assignments 314 and floor plans 316 for the associated facility.

Mandated standards include a plurality of elements of performance (EPs) to indicate what needs to be completed to satisfy the standard. An example EP could be “the fire doors need to be inspected once per year. Embodiments of the present disclosure look not only at what frequency an asset should be inspected, but also how the inspection should be performed (what should be the inspection points). Each inspection point indicates a requirement of the respective EP. For example, an inspection point can be an asset inspection, a document, or an action to be taken. More specifically, an inspection point is any requirement relating to assets, machinery, instruments and other facility optional equipment, facility condition inspections, construction status and/or inspections relating to having valid current supporting document, as they relate to environmental care, life safety, and emergency management.

If any of those inspection points is such that risk assessment is required embodiments of the present disclosure determine whether a life safety measure should be in place. As inspections are performed, the associated documentation automatically files in the right place. In addition, if an inspection result requires a work order to be created, the compliance system 300 communicates with the work order system 302 to file a work order into the work order system 302 and then tracks the work order until it is completed. For actions performed by vendors outside the system, the vendors can email their report to the system and the report can be attached to a particular EP.

Embodiments of the present disclosure can be realized using an enterprise mobility system, wherein users utilize mobile assets, such as tablets and smart phones, to interact with the data and computer programs provided via the embodiments of the present disclosure. In general, the enterprise mobility nature of the embodiments of the present disclosure allows for increased productivity and decreased expenses for the facility.

In addition, embodiments of the present disclosure can be realized via Internet-based computing delivered to the facility's computers and devices through the Internet, often referred to as cloud-based computing. This allows embodiments to be accessed and shared as virtual resources in a secure and scalable manner, which further supports enterprise mobility as described above. In some aspect, embodiments of the present disclosure can be accessed and delivered via the Internet, instead of a local hard drive. In this manner, the related infrastructure of the compliance system 300 can be maintained by a provider, instead of the health facility itself.

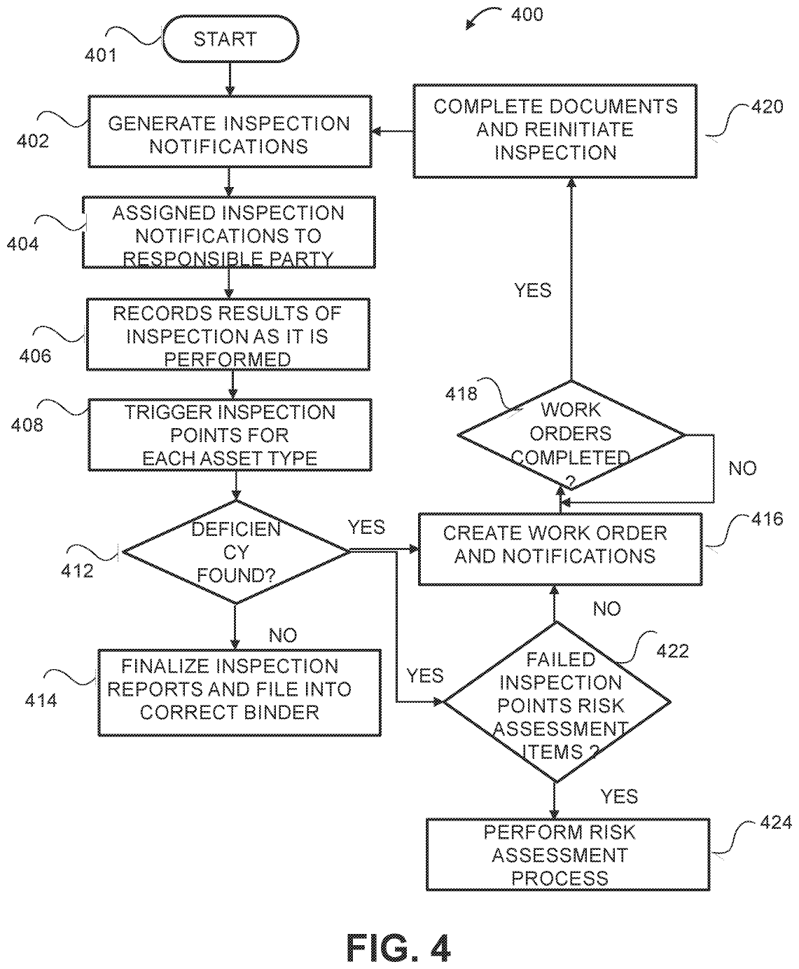

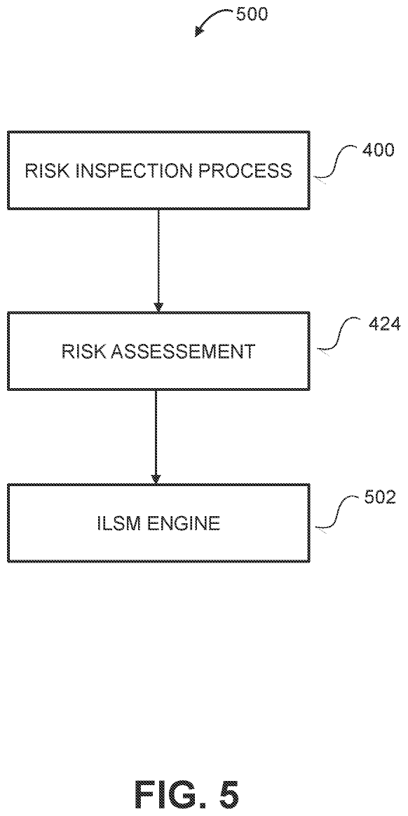

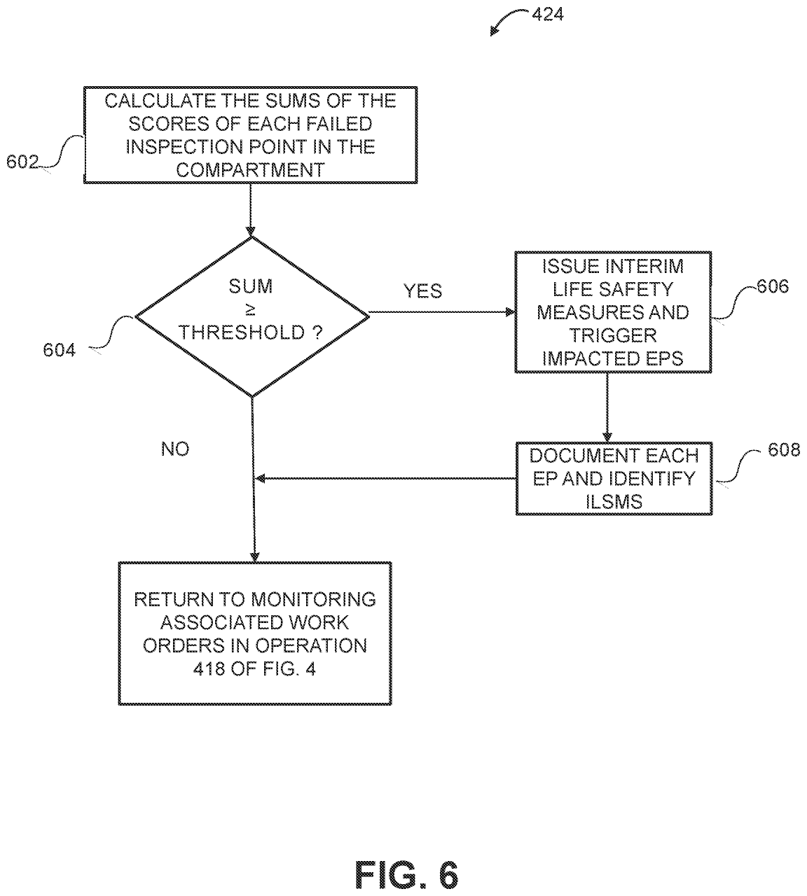

FIG. 4 illustrates an exemplary flow diagram representation of an inspection method 400 for compliance and risk assessment, in accordance with an embodiment of the present disclosure.

At step 401, initialization and set up operations are performed. Initialization and set up operations can include, for example, integrating the compliance system with a facility's work order system, providing EP and associated risk assessment scores, providing the hospital's on-going inspection frequencies, associating responsible parties with each EP, providing an interim life safety measures policy, determining vendor assignments, and uploading facility floor plans.

At step 402, inspection notifications are generated, and, at step 404, these notifications are assigned to in-house responsible party or an outside vendor. Based on a predetermined frequency, inspection notifications for EPs are generated by the compliance system. In one embodiment, the compliance system shows the user the standard requirements and the related elements of performance. Beside each EP the user is shown the mandated frequency and the specific person assigned to each EP.

At step 406, the system records the results of the inspection as it is performed. During the inspection, inspection points are triggered for each asset type, at step 408. In addition, rounds and drills are recorded with respect to whether they have been performed. Embodiments of the present disclosure analyze each requirement of the JC to determine if the requirement relates to an asset inspection, a document, or if the requirement relates to a miscellaneous action that must be performed, such as filing rounds that performed every day.

A determination is then made as to whether a deficiency has been found in any inspection point, at step 412. If no deficiency is found in any inspection point, the method 400 continues to step 414. Otherwise, the method 400 branches to step 416 and step 422.

At step 414, the inspection reports are finalized and filed into the correct binder. During this operation, the inspection reports are e-signed, and the inspection points are reset to the next inspection date. In one embodiment, work from vendors, policies that must be reviewed, etc. are all stored in a repository that is used to show the user whether they have satisfied each requirement. If any requirement is not satisfied, the user can view into the repository and view what is upcoming, what is due, what is past due, where they are now, and how does that affect them in terms of the inspection that will be performed by the JC.

In one embodiment, the compliance system has a plurality of binders. When documents are attached to the compliance system, the document is automatically filed into the appropriate binder. Items emailed to the system are sent to the inbox, as indicated in FIG. 3. From this inbox, email attachments can be attached to a particular EP. Each EP has its own document requirements indicating what the document needs to include to satisfy that EP. These requirements can include such aspects as, for example, a signature and/or a date. When a user attempts to attach a document to an EP, embodiments present a number of questions to the user based on what is required to satisfy the EP's document requirements. For the document to be accepted, the user must answer in the affirmative for each question.

If deficiencies are found in any inspection point, the method 400 branches to step 416 and step 422. In one embodiment, whenever an inspection point that relates to a risk assessment item fails inspection, the user is given a choice of whether to have “Time to Resolve” or to “Mark as deficiency.” When the user selects “Time to Resolve,” the user is given a predetermined amount of time, for example four hours, to resolve the inspection point deficiency before the inspection point failure result is transferred to steps 416 and 422. If the user resolves the deficiency within the predetermined time, the inspection point failure is not transferred, and the method moves to step 414. Otherwise, the inspection point failure is transferred to steps 416 and 422. If the user selects “Mark as deficiency,” the inspection point failure is immediately transferred to steps 416 and 422.

At step 416, a work order and notifications are created. As mentioned previously, embodiments of the present disclosure are initially integrated with a work order system. At step 416, the compliance system communicates with the work order system to create one or more work orders to correct the deficiencies found during the inspection. The compliance system then monitors these work orders to determine when they are completed.

At step 418, a determination is made as to whether the work orders are completed. If the work orders are not completed, the method 400 loops to another step 418 as it continues to monitor the work orders. If the work orders are completed the method 400 continues to step 420, where the documents are completed, and the inspection is reinitiated.

In addition to performing step 416, the method 400 branches to step 422 when deficiencies are found in any inspection point. At step 422, a decision is made as to whether any of the failed inspection points are risk assessment items. If none of the failed inspection points are risk assessment items, no additional actions are performed, and the method continues to generate work orders and monitor them as discussed above with respect to steps 416 and 418. Otherwise, a risk assessment may be performed at step 424.

FIG. 5 illustrates an exemplary block diagram representation of elements of a compliance and risk assessment computer program 500, in accordance with an embodiment of the present disclosure. The computer program 500 includes computer instructions that perform the inspection method 400 described previously with respect to FIG. 4, computer instructions that perform risk assessment 424 as described with reference to FIG. 6, and an ILSM engine 502 that issues Interim Life Safety Measures (ILSMs).

As noted above, during the inspection method 400, whenever an inspection point that relates to a risk assessment item fails inspection, the user is given a choice of whether to have “Time to Resolve” or to “Mark as deficiency.” When the user selects “Time to Resolve,” the user is given a predetermined amount of time, for example four hours, to resolve the inspection point deficiency before the inspection point failure result is transferred to risk assessment 424. If the user resolves the deficiency within the predetermined time, the inspection point failure is not transferred to risk assessment 424, otherwise the inspection point failure is transferred to risk assessment 424. If the user selects “Mark as deficiency,” the inspection point failure is immediately transferred to risk assessment process 424. In risk assessment 424, a determination is made as to whether failed points of inspection of the EP data indicate that the inspection point failure should be sent to the ILSM engine 502, where an ILSM is issued if required.

Embodiments of the present disclosure analyze each specific requirement of the standard ILSM to create the process required within each function. In addition, the facility's own ILSM processes, as determined in their own policy, can be added to the ILSM engine. In addition to creation via assets, ILSMs can also be triggered by events, such as construction. These additional events are determined during set up of the compliance and risk assessment system based on existing standards and the requirements of the particular facility using the present disclosure.

FIG. 6 illustrates an exemplary flow diagram representation of a process for risk assessment 424 in the compliance system 300, in accordance with an embodiment of the present disclosure. During an inspection, if a deficiency is found in an inspection point that is a risk assessment item, the risk assessment process 424 is performed. In the compliance system 300, particular inspection points require risk analysis, and thus are given a score based on the importance and the harm created when the inspection point fails inspection.

At step 602, all deficiencies in a particular compartment are collected and the sums of the scores of each failed inspection point in the compartment is calculated. Each facility is subdivided into a plurality of compartments. These compartments can be of any size and are usually contained within a single floor of a facility. A compartment can comprise a single room, a portion of a room, a plurality of rooms, or any other combination that facilitates risk assessment for the facility. For example, as mentioned previously, floor plans of the facility are uploaded to the system during initialization. Each floor plan indicates the location of assets on the floor and shows the location of each compartment that makes up the floor. As assets are inspected, any deficiencies within the compartment are tracked. Deficiencies for items that require risk assessment are collected for each compartment, and the sums of failed risk assessment items scores are calculated for each compartment.

It should be noted that embodiments of the present disclosure can allow the user to drag and drop assets information into an icon created which can be then dragged and dropped on to the floor plan, select an asset on the floor plan to show all the details for the asset, which comes from the integrated work order system or can be manually added. The user can then inspect the asset, since the compliance system shows all the inspection points that are relevant to that type of asset. For example, a fire door can have certain inspection points and the compliance system shows whether they comply. Some of the inspection points include a score. These inspection points are required to go through a risk assessment to determine if interim safety measures are required.

A decision is then made, at step 604, as to whether the sum of the scores of each failed inspection point in a compartment equals or exceeds a predetermined threshold for the compartment. If the sum does not equal or exceed the predetermined threshold for the compartment, no additional action is required, and the method continues to generate work orders and monitor them as discussed above with respect to steps 416 and 418. However, if the sum equals or exceeds the predetermined threshold for the compartment the method 424 branches to step 606 where one or more interim life safety measure are issued. At step 608, the method 424 includes documenting each EP and identifying ILSMs.

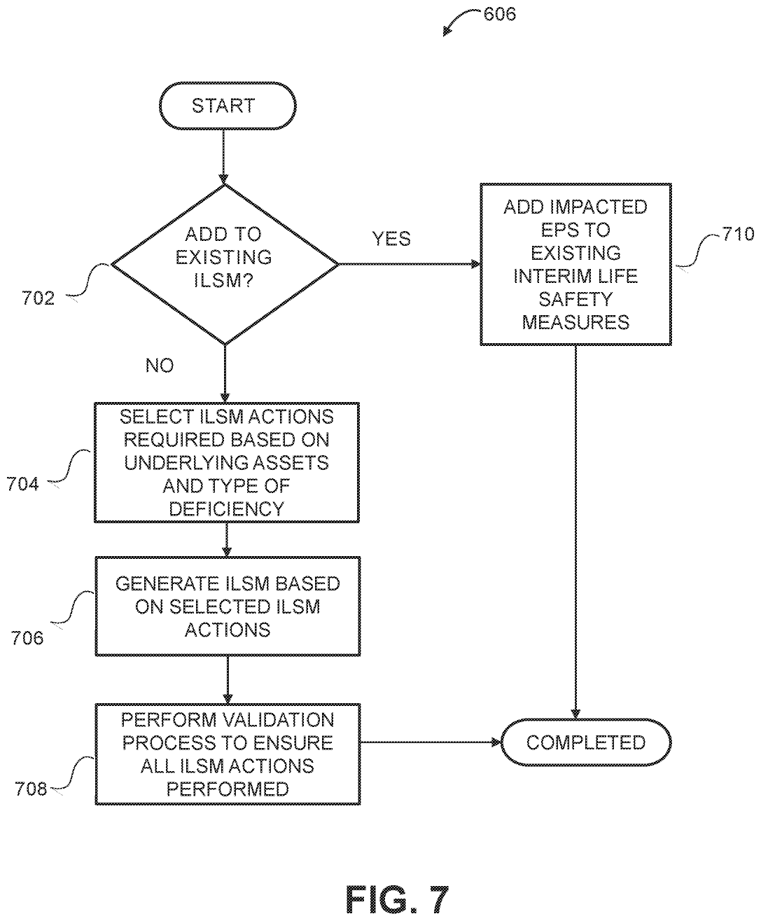

FIG. 7 illustrates an exemplary flow diagram representation of a method 606 for issuing an ILSM, in accordance with an embodiment of the present disclosure. Because an existing ILSM may already be in place, a determination is made, at step 702, as to whether to create a new ILSM or add the asset that created the ILSM to an existing ILSM. If a new ILSM is to be generated, the method 606 continues to step 704, otherwise the method 606 branches to step 710 where the impacted EPs are added to an existing ILSM.

At step 704, ILSM actions are selected based on the underlying assets and the type of deficiencies involved. Embodiments of the present disclosure store in a database a list of ILSM actions defined by accrediting bodies and the particular facility that, when put into action, protect the safety and health of patients by compensating for hazards caused by Life Safety Code deficiencies or construction activity. In addition to the list of ILSM actions, embodiments store in a database correlating relationship between the types of deficiencies that may be encountered and the ILSM actions that address these deficiencies. Embodiments of the present disclosure then automatically identify and determine, based on the asset deficiencies, ILSM actions to be taken.

Embodiments of the present disclosure then generate an ILSM based on the selected ILSM actions, in operation 706. The ILSM is a list of selected ILSM actions that are to be performed. As noted above, ILSMs are health and safety measures put in place to protect the safety of patients, visitors, and staff who work in the hospital. ILSMs can, for example, take the form of exit signs and pathways to an egress point, fire protection systems including smoke detectors, fire suppression, fire extinguishers and fire alarm systems, smoke barriers, emergency evacuation plans, in addition to many other items that contribute to the well-being and safety of occupants in the hospital or healthcare facility. Construction or maintenance activities can have an impact on the life safety systems in the hospital, thus requiring an Interim plan to address the deficiencies created by the work activity.

When an ILSM is issued, ILSM actions, which are required steps, are selected from a pre-defined list, and documented. For example, ILSM actions that may be suggested by ILSM engine might include for example:
(a) Provides temporary but equivalent fire alarm and detection systems for use when a fire system is impaired.
(b) Post signage identifying the location of alternative exits to everyone affected a picture of the posted signage can be taken and each of the item provide for the ability to take pictures and enter notes.
(c) Enforces storage, housekeeping, and debris-removal practices that reduce the building's flammable load.
(d) Enforces storage, housekeeping, and debris-removal practices that reduce the building's flammable load.
(e) debris removal
(f) Uses temporary construction partitions that are smoke-tight or made of noncombustible or limited-combustible material that will not contribute to the development or spread of fire.
(g) when the hospital identifies Life Safety Code deficiencies that cannot be immediately the hospital either evacuates the building or
(h) Initiates a fire watch and
(i) Other pre-defined items.

At step 708, a validation process is performed to ensure all ILSM actions are performed. The ILSM engine of the embodiments of the present disclosure provides the user with the ability to document each ILSM action taken and indicate as performed. Once an ILSM is created, the validation process is activated to confirm that all documents and all required action were performed. Alerts are issued if ILSM actions are not performed within the required timeframe. Alerts also are issued at each step of the process, upon deficiency discovery, upon generation of ILSM, and upon actions taken based on ILSM requirements, which provides full documentations of action item and steps taken and are reaffirmed, based on current ILSM incident duration. The ILSM engine also monitors the work order system for completion of all work orders underlying as ILSM incident. Once the work orders are completed, it allows for closing of that ILSM incident, as discussed subsequently.

Referring back to FIG. 6, during an inspection, if an item requires risk assessment the item will have a score. Embodiments of the present disclosure examine all the items which are risk assessment items, put them in a holding area, and then determine whether all those combined within a particular compartment reach a threshold.

For example, on an exemplary floor within a hospital, there exist a plurality of small compartments. Within one small compartment there may be, for example, one item that has a score of 3 and another item that has a score of 4. In this example, the compartment has a threshold of 6. In this example, since the sum of the failed risk assessment items is 7, which exceeds the compartment threshold of 6, an ILSM is issued. If only one of these are present, then an ILSM is not issued because the sum did not exceed the compartment threshold of 6.

Embodiments of the present disclosure utilized the fourteen elements of performance mandated by the JC that are required to be addressed. The compliance system of the embodiments of the present disclosure issues these items, thus requiring the user to address the items. At step 608, each of these items is required to be documented that “yes” the user has taken an action on the item. Thereafter, the process 424 continues to monitor the associated work orders in operation 418 of FIG. 4. Once those work orders are closed, the deficiencies are closed, and the ILSM is no longer required. Thereafter, the assets are posted again for re-inspection in operation 420 of FIG. 2.

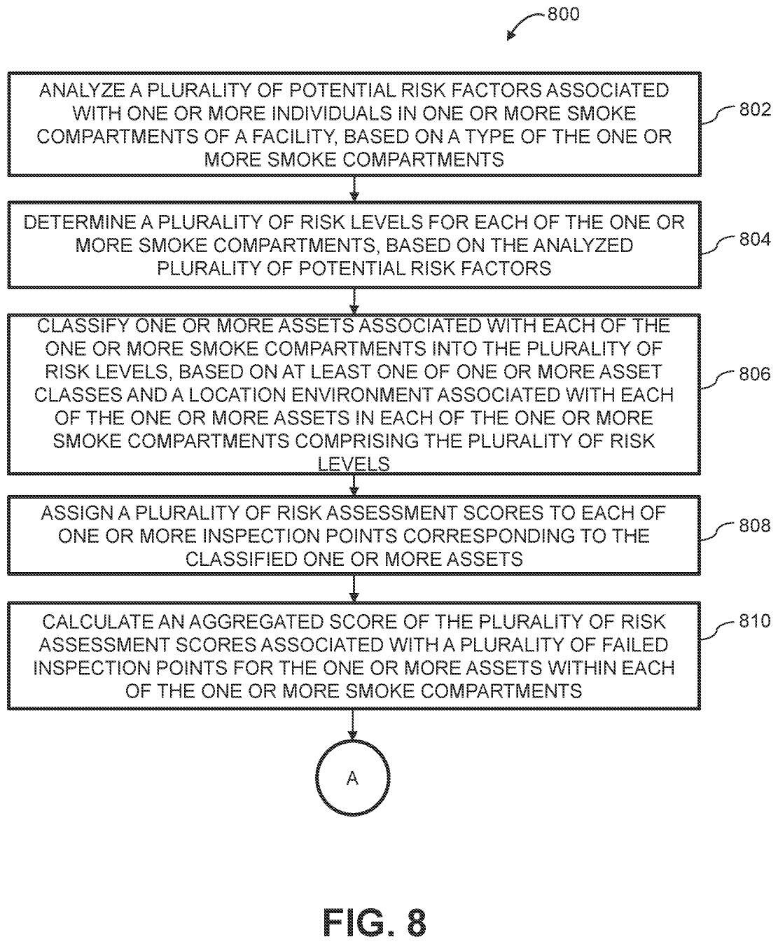

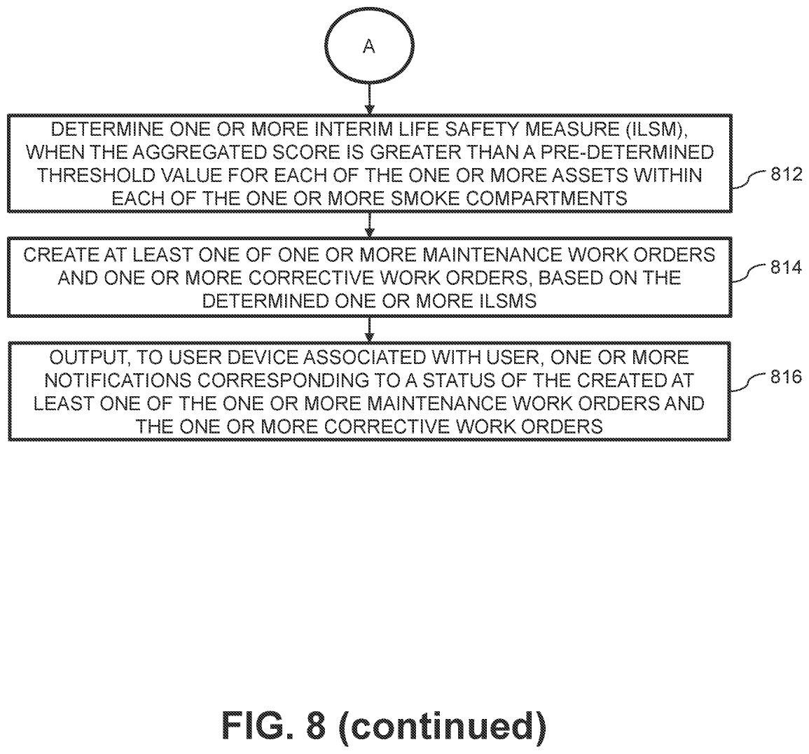

FIG. 8 illustrates a flowchart depicting a method 800 for determining an interim life safety measure (ILSM) for one or more smoke compartments in a facility, according to an example embodiment of the present disclosure.

At step 802, the method 800 may include analyzing, by the hardware processor 110 associated with a computer-implemented system 102, a plurality of potential risk factors associated with one or more individuals in the one or more smoke compartments of a facility, based on a type of the one or more smoke compartments.

At step 804, the method 800 may include determining, by the hardware processor 110, a plurality of risk levels for each of the one or more smoke compartments, based on the analyzed plurality of potential risk factors.

At step 806, the method 800 may include, in response to determining the plurality of risk levels, classifying, by the hardware processor 110, one or more assets associated with each of the one or more smoke compartments into the plurality of risk levels, based on at least one of one or more asset classes and a location environment associated with each of the one or more assets in each of the one or more smoke compartments comprising the plurality of risk levels.

At step 808, the method 800 may include, assigning, by the hardware processor 110, a plurality of risk assessment scores to each of one or more inspection points corresponding to the classified one or more assets. The risk assessment score corresponds to at least one of an importance and a potential harm created when the one or more inspection point fails an inspection. Each of the one or more inspection points corresponds to a requirement of an element of performance (EP).

At step 810, the method 800 may include calculating, by the hardware processor 110, an aggregated score of the plurality of risk assessment scores associated with a plurality of failed inspection points for the one or more assets within each of the one or more smoke compartments.

At step 812, the method 800 may include determining, by the hardware processor, one or more ILSMs, when the aggregated score is greater than a pre-determined threshold value for each of the one or more assets within each of the one or more smoke compartments. The ILSM is a health and safety measure to protect the one or more individuals at the facility.

At step 814, the method 800 may include creating, by the hardware processor 110, at least one of one or more maintenance work orders and one or more corrective work orders, based on the determined one or more ILSMs.

At step 816, the method 800 may include outputting, by the hardware processor 110, to the user device 106 associated with the user, one or more notifications corresponding to a status of the created at least one of the one or more maintenance work orders and the one or more corrective work orders. The one or more notifications informs the user to provide instructions for creating the at least one of the one or more maintenance work orders or the one or more corrective work orders and inform the user to monitor the status.

The order in which the method 800 is described is not intended to be construed as a limitation, and any number of the described method blocks may be combined or otherwise performed in any order to implement the method 800 or an alternate method. Additionally, individual blocks may be deleted from the method 800 without departing from the spirit and scope of the present disclosure described herein. Furthermore, the method 800 may be implemented in any suitable hardware, software, firmware, or a combination thereof, that exists in the related art or that is later developed. The method 800 describes, without limitation, the implementation of the system 102 and 300. A person of skill in the art will understand that method 800 may be modified appropriately for implementation in various manners without departing from the scope and spirit of the disclosure.

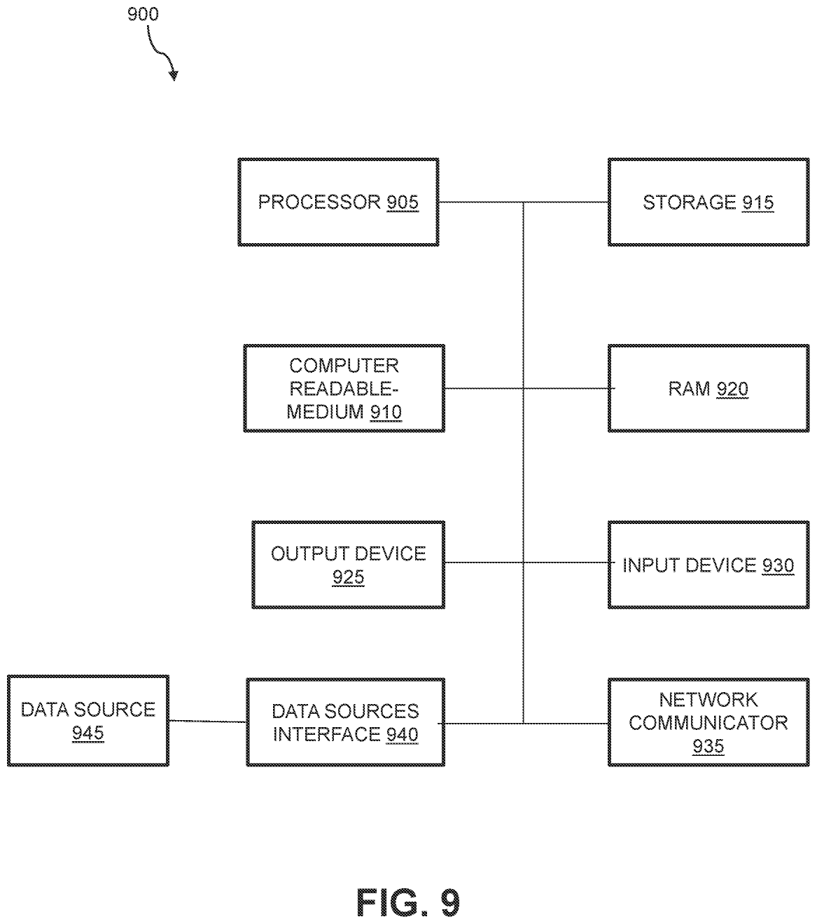

FIG. 9 illustrates an exemplary block diagram representation of a hardware platform 900 for implementation of the disclosed system 102, according to an example embodiment of the present disclosure. For the sake of brevity, the construction, and operational features of the system 102 which are explained in detail above are not explained in detail herein. Particularly, computing machines such as but not limited to internal/external server clusters, quantum computers, desktops, laptops, smartphones, tablets, and wearables which may be used to execute the system 102 or may include the structure of the hardware platform 900. As illustrated, the hardware platform 900 may include additional components not shown, and some of the components described may be removed and/or modified. For example, a computer system with multiple GPUs may be located on external-cloud platforms including Amazon Web Services, or internal corporate cloud computing clusters, or organizational computing resources.

The hardware platform 900 may be a computer system such as the system 102 that may be used with the embodiments described herein. The computer system may represent a computational platform that includes components that may be in a server or another computer system. The computer system may execute, by the processor 905 (e.g., a single or multiple processors) or other hardware processing circuit, the methods, functions, and other processes described herein. These methods, functions, and other processes may be embodied as machine-readable instructions stored on a computer-readable medium, which may be non-transitory, such as hardware storage devices (e.g., RAM (random access memory), ROM (read-only memory), EPROM (erasable, programmable ROM), EEPROM (electrically erasable, programmable ROM), hard drives, and flash memory). The computer system may include the processor 905 that executes software instructions or code stored on a non-transitory computer-readable storage medium 910 to perform methods of the present disclosure. The software code includes, for example, instructions to generate time-based contextual graph in cloud computing environment. In an example, the potential risk analyzing subsystem 206, the risk level determining subsystem 208, the asset classifying subsystem 210, the score assigning subsystem 212, the aggregated score calculating subsystem 214, the Interim Life Safety Measure (ILSM) determining subsystem 216, the work order creating subsystem 218, and the notification outputting subsystem 220, may be software codes or components performing these rules.

The instructions on the computer-readable storage medium 910 are read and stored the instructions in storage 915 or in random access memory (RAM). The storage 915 may provide a space for keeping static data where at least some instructions could be stored for later execution. The stored instructions may be further compiled to generate other representations of the instructions and dynamically stored in the RAM such as RAM 920. The processor 905 may read instructions from the RAM 920 and perform actions as instructed.

The computer system may further include the output device 925 to provide at least some of the results of the execution as output including, but not limited to, visual information to users, such as external agents. The output device 925 may include a display on computing devices and virtual reality glasses. For example, the display may be a mobile phone screen or a laptop screen. GUIs and/or text may be presented as an output on the display screen. The computer system may further include an input device 930 to provide a user or another device with mechanisms for entering data and/or otherwise interact with the computer system. The input device 930 may include, for example, a keyboard, a keypad, a mouse, or a touchscreen. Each of these output devices 925 and input device 930 may be joined by one or more additional peripherals. For example, the output device 925 may be used to display the results such as bot responses by the executable chatbot.

A network communicator 935 may be provided to connect the computer system to a network and in turn to other devices connected to the network including other clients, servers, data stores, and interfaces, for example. A network communicator 935 may include, for example, a network adapter such as a LAN adapter or a wireless adapter. The computer system may include a data sources interface 940 to access the data source 945. The data source 945 may be an information resource. As an example, a database of exceptions and rules may be provided as the data source 945. Moreover, knowledge repositories and curated data may be other examples of the data source 945.

Embodiments of the present disclosure provide a system and a method determining an interim life safety measure (ILSM) for one or more smoke compartments in a facility. The present disclosure provides a system and method that affords hospitals the ability to assure ongoing compliance and reduce cost by migrating from their current existing costly manual inspections to using a central repository and tracking solution that helps improve healthcare physical environment quality of service and provides for successful compliance outcomes. Embodiments of the present disclosure ensure Interim Life Safety Measures (ILSMs) are completed, while also ensuring and validating the required activities are performed on time. Thus, embodiments of the present disclosure schedule regulatory compliance activities around regulatory mandated timelines. Embodiments of the present disclosure also provide warnings and alarms when deadlines approach, give emergency notification to users when deadlines have not been met, and provide ongoing, comprehensive regulatory compliance reports to assist in the management of these programs. As a result, hospitals can begin a survey knowing required compliance activities have been completed on time and documented correctly, thus dramatically reducing the probability of being cited overall.

One of ordinary skill in the art will appreciate that techniques consistent with the present disclosure are applicable in other contexts as well without departing from the scope of the disclosure.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, and the like. The functions performed by various modules described herein may be implemented in other modules or combinations of other modules. For the purposes of this description, a computer-usable or computer readable medium can be any apparatus that can comprise, store, communicate, propagate, or transport the program for use by or in connection with the instruction execution system, apparatus, or device.

A description of an embodiment with several components in communication with each other does not imply that all such components are required. On the contrary, a variety of optional components are described to illustrate the wide variety of embodiments of the present disclosure. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the disclosure need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, and the like. of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the disclosure be limited not by this detailed description, but rather by any claims that issue on an application based here on. Accordingly, the embodiments of the present disclosure are intended to be illustrative, but not limiting, of the scope of the disclosure, which is set forth in the following claims.

What is claimed is:

1. A computer implemented system for determining an interim life safety measure (ILSM) for one or more smoke compartments in a facility, the computer implemented system comprising:
a hardware processor, and
a memory coupled to the hardware processor, wherein the memory comprises a set of program instructions in form of a plurality of subsystems that are configured to be executed by the hardware processor, wherein the plurality of subsystems comprises:
analyzing, by a potential risk analyzing subsystem, a plurality of potential risk factors associated with one or more individuals in one or more smoke compartments of a facility, based on a type of the one or more smoke compartments;
determining, by a risk level determining subsystem, a plurality of risk levels for each of the one or more smoke compartments, based on the analyzed plurality of potential risk factors;
in response to determining the plurality of risk levels, classifying, by an asset classifying subsystem, one or more assets associated with each of the one or more smoke compartments into the plurality of risk levels, based on at least one of one or more asset classes and a location environment associated with each of the one or more assets in each of the one or more smoke compartments comprising the plurality of risk levels;
assigning, by a score assigning subsystem, a plurality of risk assessment scores to each of one or more inspection points corresponding to the classified one or more assets, wherein the risk assessment score corresponds to at least one of an importance and a potential harm created when the one or more inspection point fails an inspection, and wherein each of the one or more inspection points corresponds to a requirement of an element of performance (EP);
calculating, by an aggregated score calculating subsystem, an aggregated score of the plurality of risk assessment scores associated with a plurality of failed inspection points for the one or more assets within each of the one or more smoke compartments;
determining, by an Interim Life Safety Measure (ILSM) determining subsystem, one or more ILSMs, when the aggregated score is greater than a pre-determined threshold value for each of the one or more assets within each of the one or more smoke compartments, wherein the ILSM is a health and safety measure to protect the one or more individuals at the facility;
creating, by a work order creating subsystem, at least one of one or more maintenance work orders and one or more corrective work orders, based on the determined one or more ILSMs; and
outputting, by a notification outputting subsystem, to a user device associated with a user, one or more notifications corresponding to a status of the created at least one of the one or more maintenance work orders and the one or more corrective work orders, wherein the one or more notifications inform the user to provide instructions for creating the at least one of the one or more maintenance work orders or the one or more corrective work orders, and inform the user to monitor the status.

2. The computer implemented system of claim 1, wherein the plurality of subsystems further comprises:
determining, by the work order creating subsystem, the status of at least one of the one or more maintenance work orders is indicated as not completed and the status of the one or more corrective work orders comprises one or more reported deficiencies requiring an immediate corrective action;
retrieving, by a profile retrieving subsystem, from a database, a risk profile corresponding to the location environment associated with each of the one or more assets in each of the one or more smoke compartments, wherein the risk profile comprises at least one of the potential risk factors, the plurality of risk levels, plurality of risk assessment scores, and the aggregated score; and
prioritizing, by the work order creating subsystem, each of at least of the one or more maintenance work orders and the one or more corrective work orders, based on the risk profile.

3. The computer implemented system of claim 1, wherein the plurality of subsystems further comprises:
determining, by a failed inspection determining subsystem, when the failed inspection points correspond to risk assessment assets, the location environment of the one or more assets corresponding to at least one of one or more supplementary facilities and one or more third party vendors, and a proximity to a subsequently discovered risk assessment assets; and
incrementing, by the score assigning subsystem, the risk assessment score, in response to the determined risk assessment assets impacting the aggregated risk assessment score.

4. The computer implemented system of claim 1, wherein the plurality of subsystems further comprises:
storing, by an action storing subsystem, in the database, a plurality of ILSM actions and a plurality of correlating deficiency assets, wherein each ILSM action defines an action related to protecting the individuals at the facility, and wherein each correlating deficiency asset defines a correlation between a deficiency encountered at the facility and at least one ILSM action.

5. The computer implemented system of claim 4, wherein the plurality of subsystems further comprises:
selecting, by an action selecting subsystem, a plurality of the plurality of ILSM actions based on deficiencies related to the failed inspection points and related correlating deficiency items.

6. The computer implemented system of claim 5, wherein the ILSM is determined by generating a list of the selected ILSM actions.

7. The computer implemented system of claim 1, wherein the plurality of subsystems further comprises:
generating, by the notification outputting subsystem, the one or more notifications that the determined ILSM is no longer needed when at least one of the one or more work orders and the one or more corrective work orders associated with the generated ILSM are completed.

8. The computer implemented system of claim 1, wherein the plurality of risk levels are comprised of one or more dynamic values for each of the one or more asset classes, based on the location environment associated with each of the one or more assets.

9. The computer implemented system of claim 1, wherein the risk assessment is not performed for a failed inspection point when the risk assessment score associated with the failed inspection point is zero.

10. The computer implemented system of claim 1, wherein the risk assessment for a failed inspection point is delayed for a predetermined time when a “time to resolve” option is selected for a failed inspection point, and wherein risk assessment for the failed inspection point is performed when the failed inspection point is not completed within the predefined time.

11. The computer implemented system of claim 1, wherein the plurality of potential risk factors are analyzed using at least one of an artificial intelligence (AI) based smoke detectors, a predictive maintenance based AI model, a real-time monitoring based AI model, an emergency response based AI model, and an occupant tracking based AI model, and
wherein the one or more asset class are classified using at least one of an asset class inheritance-based AI models and an asset class acquisition-based AI models.

12. A computer implemented method for determining an interim life safety measure (ILSM) for one or more smoke compartments in a facility, the computer implemented method comprising:
analyzing, by a hardware processor associated with a computer-implemented system, a plurality of potential risk factors associated with one or more individuals in one or more smoke compartments of a facility, based on a type of the one or more smoke compartments;
determining, by the hardware processor, a plurality of risk levels for each of the one or more smoke compartments, based on the analyzed plurality of potential risk factors;
in response to determining the plurality of risk levels, classifying, by the hardware processor, one or more assets associated with each of the one or more smoke compartments into the plurality of risk levels, based on at least one of one or more asset classes and a location environment associated with each of the one or more assets in each of the one or more smoke compartments comprising the plurality of risk levels;
assigning, by the hardware processor, a plurality of risk assessment scores to each of one or more inspection points corresponding to the classified one or more assets, wherein the risk assessment score corresponds to at least one of an importance and a potential harm created when the one or more inspection point fails an inspection, and wherein each of the one or more inspection points corresponds to a requirement of an element of performance (EP);
calculating, by the hardware processor, an aggregated score of the plurality of risk assessment scores associated with a plurality of failed inspection points for the one or more assets within each of the one or more smoke compartments;
determining, by the hardware processor, one or more ILSMs, when the aggregated score is greater than a pre-determined threshold value for each of the one or more assets within each of the one or more smoke compartments, wherein the ILSM is a health and safety measure to protect the one or more individuals at the facility;
creating, by the hardware processor, at least one of one or more maintenance work orders and one or more corrective work orders, based on the determined one or more ILSMs; and
outputting, by the hardware processor, to the user device associated with a user, one or more notifications corresponding to a status of the created at least one of the one or more maintenance work orders and the one or more corrective work orders, wherein the one or more notifications inform the user to provide instructions for creating the at least one of the one or more maintenance work orders or the one or more corrective work orders, and inform the user to monitor the status.

13. The computer implemented method of claim 12 further comprising:
determining, by the hardware processor, the status of at least one of the one or more maintenance work orders is indicated as not completed and the status of the one or more corrective work orders comprises one or more reported deficiencies requiring an immediate corrective action;
retrieving, by the hardware processor, from a database, a risk profile corresponding to the location environment associated with each of the one or more assets in each of the one or more smoke compartments, wherein the risk profile comprises at least one of the potential risk factors, the plurality of risk levels, plurality of risk assessment scores, and the aggregated score; and
prioritizing, by the hardware processor, each of at least of the one or more maintenance work orders and the one or more corrective work orders, based on the risk profile.

14. The computer implemented method of claim 12 further comprising:
determining, by the hardware processor, when the failed inspection points correspond to risk assessment assets, the location environment of the one or more assets corresponding to at least one of one or more supplementary facilities and one or more third party vendors, and a proximity to a subsequently discovered risk assessment assets; and
incrementing, by the hardware processor, the risk assessment score, in response to the determined risk assessment assets impacting the aggregated risk assessment score.

15. The computer implemented method of claim 12 further comprising:
storing, by the hardware processor, in the database, a plurality of ILSM actions and a plurality of correlating deficiency assets, wherein each ILSM action defines an action related to protecting the individuals at the facility, and wherein each correlating deficiency asset defines a correlation between a deficiency encountered at the facility and at least one ILSM action.

16. The computer implemented method of claim 15 further comprising:
selecting, by the hardware processor, a plurality of the plurality of ILSM actions based on deficiencies related to the failed inspection points and related correlating deficiency items, wherein the ILSM is determined by generating a list of the selected ILSM actions.

17. The computer implemented method of claim 12 further comprising:
generating, by the hardware processor, the one or more notifications that the determined ILSM is no longer needed when at least one of the one or more work orders and the one or more corrective work orders associated with the generated ILSM are completed.

18. The computer implemented method of claim 12, wherein the plurality of risk levels are comprised of one or more dynamic values for each of the one or more asset classes, based on the location environment associated with each of the one or more assets.

19. The computer implemented method of claim 12, wherein the risk assessment is not performed for a failed inspection point when the risk assessment score associated with the failed inspection point is zero.

20. The computer implemented method of claim 12, wherein the risk assessment for a failed inspection point is delayed for a predetermined time when a “time to resolve” option is selected for a failed inspection point, and wherein risk assessment for the failed inspection point is performed when the failed inspection point is not completed within the predefined time.

21. The computer implemented method of claim 12, wherein the plurality of potential risk factors is analyzed using at least one of an artificial intelligence (AI) based smoke detectors, a predictive maintenance based AI model, a real-time monitoring based AI model, an emergency response based AI model, and an occupant tracking based AI model, and
wherein the one or more asset class are classified using at least one of an asset class inheritance-based AI models and an asset class acquisition-based AI models.

22. A non-transitory computer-readable medium comprising machine-readable instructions that are executable by a hardware processor to:
analyze a plurality of potential risk factors associated with one or more individuals in one or more smoke compartments of a facility, based on a type of the one or more smoke compartments;
determine a plurality of risk levels for each of the one or more smoke compartments, based on the analyzed plurality of potential risk factors;
in response to determining the plurality of risk levels, classify one or more assets associated with each of the one or more smoke compartments into the plurality of risk levels, based on at least one of one or more asset classes and a location environment associated with each of the one or more assets in each of the one or more smoke compartments comprising the plurality of risk levels;
assign a plurality of risk assessment scores to each of one or more inspection points corresponding to the classified one or more assets, wherein the risk assessment score corresponds to at least one of an importance and a potential harm created when the one or more inspection point fails an inspection, and wherein each of the one or more inspection points corresponds to a requirement of an element of performance (EP);
calculate an aggregated score of the plurality of risk assessment scores associated with a plurality of failed inspection points for the one or more assets within each of the one or more smoke compartments;
determine one or more Interim Life Safety Measure (ILSM), when the aggregated score is greater than a pre-determined threshold value for each of the one or more assets within each of the one or more smoke compartments, wherein the ILSM is a health and safety measure to protect the one or more individuals at the facility;
create at least one of one or more maintenance work orders and one or more corrective work orders, based on the determined one or more ILSMs; and
output, to the user device associated with a user, one or more notifications corresponding to a status of the created at least one of the one or more maintenance work orders and the one or more corrective work orders, wherein the one or more notifications inform the user to provide instructions for creating the at least one of the one or more maintenance work orders or the one or more corrective work orders, and inform the user to monitor the status.
